(19) United States
(12) Patent Application Publication
(10) Pub. No.: US 2026/0296608 A1
(43) Pub. Date: Oct. 1, 2026

(54) APPARATUSES AND SYSTEMS FOR ARTIFACT REDUCTION IN ELECTRODERMAL ACTIVITY

(71) Applicant: University of Connecticut, Farmington, CT (US)

(72) Inventors: Ki Chon, Mansfield, CT (US); Md Billal Hossain, Willington, CT (US); Hugo Posada-Quintero, Vernon, CT (US)

(21) Appl. No.: 19/701,313

(22) Filed: Jun. 8, 2026

Related U.S. Application Data
(63) Continuation-in-part of application No. 18/313,734, filed on May 8, 2023.
(60) Provisional application No. 63/340,272, filed on May 10, 2022.

(51) Int. Cl. B63C 11/02; A61B 5/00; A61B 5/0531; A62B 9/00

(52) U.S. Cl. CPC B63C 11/02; A61B 5/0531; A61B 5/4094; A61B 5/7207; A61B 5/7267; A61B 5/7275; A61B 5/746; A62B 9/006; A61B 2503/10; B63C 2011/021

(57) ABSTRACT

Methods, systems, non-transitory computer-readable media, and apparatuses are described for predicting a health condition of a subject. An apparatus may be configured to receive a physiological signal associated with the subject. The physiological signal may include artifacts. A modified physiological signal may be generated based on an application of a machine learning model to the physiological signal. The modified physiological signal may include the physiological signal with a reduction of the artifacts. A physiological measurement may be determined based on the modified physiological signal. The health condition may be determined based on a change in the physiological measurement satisfying a threshold. The apparatus may cause an output of an indication associated with the health condition.

### CROSS REFERENCE TO RELATED PATENT APPLICATIONS

This Application is a continuation-in-part of U.S. application Ser. No. 18/313,734, filed May 8, 2023, which claims priority to U.S. Provisional Application No. 63/340,272, filed May 10, 2022, the contents of which are herein incorporated by reference in their entirety.

### FEDERALLY SPONSORED RESEARCH

This invention was made with government support under N00014-21-1-2255, awarded by the Office of Naval Research. The government has certain rights in the invention.

### BACKGROUND

This application generally relates to reducing artifacts in an electrodermal activity (EDA) signal and determining a health condition of a subject based on the EDA signal. Decompression sickness (DCS) in divers is caused by the formation of nitrogen bubbles in the blood or body tissues when there is sudden reduction in ambient pressure during the ascent phase of a dive. During a prolonged hyperbaric exposure, intake gases dissolve in the blood, then emerge from the solution as bubbles in body tissues during decompression. Symptoms of DCS may range from mild (e.g., skin itching, muscle pain, nausea, and tingling) to serious including neurological dysfunctions, spinal cord injury, cardiopulmonary collapse and disseminated intravascular coagulation. An effective way to mitigate DCS in certain situations is hyperbaric oxygen (HBO₂) pre-breathing under water. Unfortunately, breathing HBO₂ creates a risk for divers to develop central nervous system oxygen toxicity (CNS-OT). The symptoms of CNS-OT include headache, diaphoresis, nausea, tinnitus, lip twitching, tingling of the limbs, or even more serious symptoms such as seizure or loss of consciousness. Although, oxygen-induced seizures are not life-threatening in the controlled, dry environment of a hyperbaric chamber, losing consciousness or convulsing under water could result in the dislodgement of the diver's air supply from his or her mouth, and likely lead to drowning.

Studies involving animal and human models breathing HBO₂ in a pressurized chamber have shown consistent seizure activities due to CNS-OT induced by HBO₂. Physiologically, augmented seizure activity is associated with elevated sympathetic activity. Therefore, a sensitive measure of sympathetic activity can be used as a surrogate physiomarker of seizure detection and more importantly seizure prediction. For example, electrodermal activity (EDA) has been used as a measure of sympathetic activity, especially due to the use of wearable technologies that may be used to monitor EDA. Conventional methods of analyzing EDA have been confined to the time domain, decomposing the signal into tonic and phasic EDA. The tonic EDA is typically quantified using the skin conductance level (SCL), the mean value of the tonic component. The phasic EDA is comprised of the skin conductance responses (SCRs). The SCRs are the rapid transient events contained in the EDA signal. However, it has been shown that there is a low reproducibility of these time-domain measures.

Generally, the term “electrodermal activity (EDA)” refers to a property of the human body that causes continuous variation in the electrical characteristics of the skin. Historically, EDA has also been known as skin conductance, galvanic skin response (GSR), electrodermal response (EDR), psychogalvanic reflex (PGR), skin conductance response (SCR), sympathetic skin response (SSR) and skin conductance level (SCL). The long history of research into the active and passive electrical properties of the skin by a variety of disciplines has resulted in an excess of names, now standardized to electrodermal activity (EDA). The traditional theory of EDA holds that skin resistance varies with the state of sweat glands in the skin. Sweating is controlled by the sympathetic nervous system, and skin conductance is an indication of psychological or physiological arousal. If the sympathetic branch of the autonomic nervous system is highly aroused, then sweat gland activity also increases, which in turn increases skin conductance. In this way, skin conductance can be a measure of emotional and sympathetic responses. More recent research and additional phenomena (resistance, potential, impedance, electrochemical skin conductance, and admittance, sometimes responsive and sometimes apparently spontaneous) suggest that EDA is complex and can be difficult to measure accurately.

For example, accurate measurement of EDA data is often hampered by motion artifacts, especially EDA data obtained via wearable technologies. These artifacts can be severe enough to cause the data to be unreliable, or corrupted, and thus, unusable. One conventional method of removing motion artifacts from the EDA data involves detecting and discarding corrupted data segments. However, this conventional method can be quite costly as the amount of usable data can be significantly reduced. Another conventional method involves the use of an unsupervised scheme using a one-class support vector machine and k-nearest neighbor distance to automatically detect motion artifacts. However, this conventional method has yet to be successfully applied to EDA data that have more dynamics. Other conventional methods involve the use of exponential smoothing and low pass filtering to remove motion artifacts from the EDA data. However, these conventional methods are non-adaptive, fail to remove artifacts with high intensity, and are subject to distorting the skin conductance response especially in portions of the data segments without artifacts. Conventional autoencoder methods involve an unsupervised artificial neural network to learn efficient coding of unlabeled data, which is then validated and refined to reproduce the original input. However, if the model is sufficiently large with many parameters to tune, it may learn an identity function so that the output is always equal to the input, and thus, the network may not create a useful representation of the data.

What are needed are techniques for reducing motion artifacts in an electrodermal activity (EDA) signal and determining a health condition of a subject based on the EDA signal. Preferably, the techniques result in rapid delivery of accurate results. Techniques should be efficient computing wise, and therefore available for implementation in low computing power systems, such as in wearable technology.

### SUMMARY

It is understood that both the following general description and the following detailed description are exemplary and explanatory only and are not restrictive. Methods, systems, and apparatuses for improved artifact reduction in electrodermal activity (EDA) and for improved health condition determinations of a subject based on EDA are described.

In some embodiments, an apparatus for predicting a health condition of a subject is provided. The apparatus may include one or more processors and a memory storing processor-executable instructions that, when executed by the one or more processors. The apparatus may be configured to receive a physiological signal associated with the subject. The physiological signal may include artifacts. A modified physiological signal may be generated based on an application of a machine learning model to the physiological signal. The modified physiological signal may include the physiological signal with a reduction of the artifacts. A physiological measurement may be determined based on the modified physiological signal. The health condition may be determined based on a change in the physiological measurement satisfying a threshold. The apparatus may cause an output of an indication associated with the health condition.

In some embodiments, one or more non-transitory computer-readable media storing processor-executable instructions for predicting a health condition of a subject are provided. The processor-executable instructions may cause the at least one processor to receive a physiological signal associated with the subject by a computing device when executed by at least one processor. The physiological signal may include artifacts. A modified physiological signal may be generated based on an application of a machine learning model to the physiological signal. The modified physiological signal may include the physiological signal with a reduction of the artifacts. A physiological measurement may be determined based on the modified physiological signal. The health condition may be determined based on a change in the physiological measurement satisfying a threshold. The processor-executable instructions may cause an output of an indication associated with the health condition.

In an embodiment, are systems for predicting a health condition of a subject associated with prolonged exposure to hyperbaric oxygen (HBO₂). The system may include a senor affixed to a surface of skin of the subject, a display configured to output an interface to the subject, and a computing device in communication with the sensor and the display. The sensor may be configured to determine a physiological signal associated with the subject based on measuring a conductance associated with the surface of skin of the subject. The display may be configured to output an interface to the subject. The computing device may be configured to receive the physiological signal from the sensor. The physiological signal may include artifacts. The physiological signal may be provided to a machine learning model. A modified physiological signal may be generated based on the machine learning model. The modified physiological signal may include the physiological signal with a reduction of the artifacts. A physiological measurement may be determined based on the modified physiological signal. A change in the physiological measurement may be determined to satisfy a threshold. The change may be caused by stress based on breathing performed by the subject during prolonged exposure to HBO₂. The health condition may be determined based on the change in the physiological measurement satisfying the threshold. The computing device may be configured to cause an output of an indication of the health condition via the interface to the subject.

Additional advantages will be set forth in part in the description which follows or may be learned by practice. The advantages will be realized and attained by means of the elements and combinations particularly pointed out in the appended claims. It is to be understood that both the foregoing general description and the following detailed description are exemplary and explanatory only and are not restrictive.

### BRIEF DESCRIPTION OF THE DRAWINGS

The accompanying drawings serve to explain the principles of the methods, apparatuses, and systems described herein:

FIG. 1 shows an example system;

FIG. 2 shows an example system environment;

FIG. 3 shows an example machine learning system;

FIG. 4 shows a flowchart of an example training method;

FIGS. 5A-5B show example physiological measurements;

FIG. 6 shows a flowchart of an example method;

FIG. 7 shows a block diagram of a computing device for implementing the example methods;

FIG. 8 shows a wrist-worn monitor;

FIG. 9 shows a monitoring vest;

FIG. 10 shows a monitoring strap;

FIG. 11 shows a rebreather system;

FIG. 12 shows a hyperbaric treatment system;

FIG. 13 shows closed-loop control of oxygen concentration in a diver or subject; and

FIG. 14 shows a flowchart for a method for physiological monitoring for bounded real-time prediction of central nervous system oxygen toxicity (CNS-OT) of a diver during hyperbaric oxygen exposure.

### DETAILED DESCRIPTION

Methods, systems, and apparatuses are described for reducing artifacts in an electrodermal activity (EDA) signal and for determining a health condition based on the EDA signal. The artifacts may be caused by one or more of motion artifacts arising from variable conduction signals as a subject moves or electronic noise arising from radiofrequency and magnetic interference. A deep convolutional autoencoder network (DCAE) may be applied to an EDA signal associated with a subject to reduce artifacts in the EDA signal due to movements performed by the subject and produce a modified EDA signal. The DCAE may be trained based on one or more training data sets associated with one or more EDA signals associated with one or more subjects. The modified EDA signal may be used to determine a time-invariant and/or a time-variant spectral analysis of the EDA signal (TVSymp). A health condition may be determined based on the TVSymp. For example, the health condition may be determined based on a change in the TVSymp satisfying a threshold. The change may be caused by stress experienced by the subject based on breathing performed by the subject during prolonged exposure to hyperbaric oxygen (HBO₂). The health condition may include one or more of a risk of seizure, a risk of central nervous system oxygen toxicity (CNS-OT), or symptoms of CNS-OT. The health condition may be output via an interface to the subject.

Generally, as discussed herein, the term “physiological signal” refers to signals obtained from a subject such as electrodermal activity (EDA) and other related or associated quantities.

Generally, as discussed herein, the term “artifact” refers to spurious contributions to meaningful data that may arise from, for example, motion noise and/or electronic noise. Motion artifacts may arise from, for example, variable conduction of signals as the subject moves about. Electronic noise may arise from, for example, radiofrequency and magnetic interference.

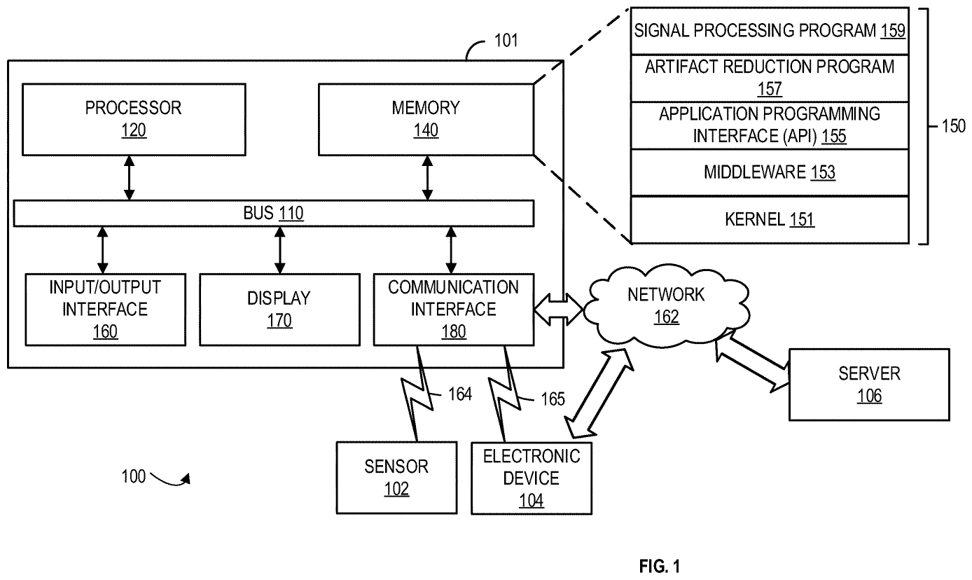

FIG. 1 shows an example system 100 reducing artifacts in an EDA signal associated with a subject and determining a health condition of the subject based on the EDA signal. For example, a time-invariant and/or a time-variant spectral analysis of the EDA signal (TVSymp) may be determined based on the EDA signal. A change in the TVSymp may be used to determine a health condition (e.g., a risk of seizure, a risk of CNS-OT, or symptoms of CNS-OT) associated the subject during prolonged exposure to HBO₂. The system 100 may include a device 101 that may be configured to use one or more methods for reducing artifacts in the EDA signal to facilitate the determination of the health condition. The device may be in communication with one or more sensors 102, one or more electronic devices 104, and one or more servers 106. The device may include a bus 110, one or more processors 120, a memory 140, an input/output interface 160, a display 170, and a communication interface 180. In a certain examples, the device 101 may omit at least one of the aforementioned elements or may additionally include other elements. The device 101 may include a wearable device, a smart watch, tablet computer, a laptop computer, a desktop computer, and the like.

The bus 110 may include a circuit for connecting the bus 110, the one or more processors 120, the memory 140, the input/output interface 160, the display 170, and/or the communication interface 180 to each other and for delivering communication (e.g., a control message and/or data) between the bus 110, the one or more processors 120, the memory 140, the input/output interface 160, the display 170, and/or the communication interface 180.

The one or more processors 120 may include one or more of a Central Processing Unit (CPU), an Application Processor (AP), or a Communication Processor (CP). The one or more processors 120 may control, for example, at least one of the bus 110, the memory 140, the input/output interface 160, the display 170, and/or the communication interface 180 of the device 101 and/or may execute an arithmetic operation or data processing for communication. For example, the one or more processors 120 may drive (e.g., cause) the display 170 and/or a speaker to issue visual and/or audio instructions or signals, respectively to an operator (e.g., subject) of the device 101, such as an indication of a health condition of the subject. For example, the health condition may include one or more of a risk of seizure, a risk of CNS-OT, or symptoms of CNS-OT. The processing (or controlling) operation of the one or more processors 120 according to various embodiments is described in detail with reference to the following drawings.

The processor-executable instructions executed by the one or more processor 120 may be stored and/or maintained by the memory 140. The memory 140 may include a volatile and/or non-volatile memory. The memory 140 may include random-access memory (RAM), flash memory, solid state or inertial disks, or any combination thereof. The memory 140 may store, for example, a command or data related to at least one of the bus 110, the one or more processors 120, the memory 140, the input/output interface 160, the display 170, and/or the communication interface 180 of the device 101. According to various examples, the memory 140 may store software and/or a program 150. For example, the program 150 may include a kernel 151, a middleware 153, an Application Programming Interface (API) 155, an artifact reduction program 157, and/or a signal processing program 159, or the like, configured for controlling one or more functions of the device 101 and/or an external device (e.g., sensor 102 or electronic device 104). At least one part of the kernel 151, middleware 153, or API 155 may be referred to as an Operating System (OS). The memory 140 may include a computer-readable recording medium (e.g., a non-transitory computer-readable medium) having a program recorded therein to perform the methods according to various embodiments by the one or more processors 120.

The kernel 151 may control or manage, for example, system resources (e.g., the bus 110, the processor 120, the memory 140, etc.) used to execute an operation or function implemented in other programs (e.g., the middleware 153, the API 155, the artifact reduction program 157, or the signal processing program 159). Further, the kernel 151 may provide an interface capable of controlling or managing the system resources by accessing individual elements of the device 101 in the middleware 153, the API 155, the artifact reduction program 157, or the signal processing program 159.

The middleware 153 may perform, for example, a mediation role, so that the API 155, the artifact reduction program 157, and/or the signal processing program 159 can communicate with the kernel 151 to exchange data. Further, the middleware 153 may handle one or more task requests received from the artifact reduction program 157 and/or the signal processing program 159 according to a priority. For example, the middleware 153 may assign a priority of using the system resources (e.g., the bus 110, the one or more processors 120, or the memory 140) of the device 101 to at least one of the artifact reduction program 157 and/or the signal processing program 159. For example, the middleware 153 may process the one or more task requests according to the priority assigned to at least one of the application programs, and thus, may perform scheduling or load balancing on the one or more task requests.

The API 155 may include at least one interface or function (e.g., instruction), for example, for file control, window control, video processing, and/or character control, as an interface capable of controlling a function provided by the artifact reduction program 157 and/or the signal processing program 159 in the kernel 151 or the middleware 153.

In an example, the artifact reduction program 157 and the signal processing program 159 may be independent of each other or integrally combined, in whole or in part.

The artifact reduction program 157 may include logic (e.g., hardware, software, firmware, etc.) that may be implemented to perform the reduction of artifacts in a physiological signal of a subject received from the sensors 102. For example, the physiological signal may include an electrodermal activity (EDA) signal. For example, the artifact reduction program 157 may be used to reduce the artifacts in the EDA signal to enable the signal processing program 159 to process a clean EDA signal (e.g., with a reduction of artifacts) to determine a health condition of the subject. For example, the artifact reduction program 157 may provide a physiological signal received from the sensors 102 to a machine learning model. The machine learning model may include a deep convolutional autoencoder (DCAE) network. The deep convolutional autoencoder network may generate a modified physiological signal. The modified physiological signal may include the physiological signal with a reduction of the artifacts. The machine learning model may be trained based on one or more training data sets. As an example, a training data set may include physiological signals from a public data set associated with physiological stimuli based on one or more of an auditory task, pain induced by electrical stimulation, a visual detection task, fear conditioning tasks, being shown aversive or neutral pictures, being shown pictures while subjected to auditory distractors, or being shown pictures of facial expressions. As an example, a training data set may include physiological signals associated with a prevalence of artifacts. As an example, a training data set may include physiological signals associated with one or more subjects experiencing central nervous system oxygen toxicity (CNS-OT) conditions. As an example, a training data set may include physiological signals associated with a first sub data set that may include physiological signals associated with a reduction of artifacts collected from both hands of one or more subjects and a second sub data set that may include physiological signals associated with a prevalence of artifacts collected from a first hand of one or more subjects and physiological signals associated with a reduction of artifacts collected from a second hand of one or more subjects.

The signal processing program 159 may include logic (e.g., hardware, software, firmware, etc.) that may determine a health condition of the subject based on the modified physiological signal. The health condition may include one or more of a risk of seizure, a risk of CNS-OT, or symptoms of CNS-OT. For example, the signal processing program 159 may be configured to determine a physiological measurement based on the modified physiological signal. The physiological measurement may include a time-invariant and/or a time-variant spectral analysis of the EDA signal (TVSymp). The signal processing program 159 may determine the health condition based on a change in the physiological measurement (e.g., TVSymp). For example, the change may be caused by stress experience by the subject based on breathing performed by the subject during prolonged exposure to hyperbaric oxygen (HBO₂). For example, the health condition may be determined based on the change in the physiological measurement (e.g., TVSymp) satisfying a threshold. The change may include an increase in the phasic components of the EDA. The increase in the phasic components of the EDA may cause the physiological measurement (e.g., TVSymp) to increase. In an example, the signal processing program 159 may first determine that the physiological measurement (e.g., TVSymp) satisfies a condition. For example, the condition may include whether the change in the physiological measurement (e.g., TVSymp) is based on an autonomic induced elevation of a phasic component of the EDA signal or a non-autonomic induced elevation of a phasic component of the EDA signal. The signal processing program 159 may determine that a change in the physiological measurement (e.g., TVSymp) satisfies a threshold, and thus, indicating a health condition of the subject, based on the physiological measurement (e.g., TVSymp) satisfying the condition. In an example, the determination of whether the physiological measurement (e.g., TVSymp) satisfies the condition may be based on an application of a machine learning model to the physiological measurement (e.g., TVSymp). The signal processing program 159 may cause the device 101 to output an indication associated with the health condition to the display 170 to be displayed to the user (e.g., subject). In an example, the signal processing program 159 may cause the device 101 to output the indication to a server 106. The server 106 may be configured to contact emergency services based on the indication. In an example, the signal processing program 159 may cause the device 101 to output the physiological signal and/or the physiological measurement to the server 106 to be saved in a database (e.g., as one or more files). The database (e.g., server 106) may group the physiological signal and/or the physiological measurement based on the user (e.g., subject).

The input/output interface 160 may include an interface for delivering an instruction or data input from a subject (e.g., an operator of the device 101) or a different external device to the different elements of the device 101. Further, the input/output interface 160 may output an instruction or data received from one or more elements of the device 101 to one or more external devices.

The display 170 may include various types of displays, for example, a Liquid Crystal Display (LCD) display, a Light Emitting Diode (LED) display, an Organic Light-Emitting Diode (OLED) display, a MicroElectroMechanical Systems (MEMS) display, or an electronic paper display. In an example, the display 170 may include a head mounted display device or a heads up display device. For example, divers may utilize dive masks while diving, especially when deep sea diving. The display 170 may be configured into the dive mask as a heads up display for displaying an interface via a window of the dive mask. The display 170 may display, for example, a variety of contents (e.g., text, image, video, icons, symbols, etc.) to the user (e.g., subject). For example, the display 170 may be configured to output an indication associated with the health condition to the user (e.g., subject). For example, the indication may include an alert notifying the user (e.g., subject) of a possible impending seizure or other health conditions such as a risk of CNS-OT, or that the user (e.g., subject) may be experiencing symptoms of CNS-OT.

The communication interface 180 may establish, for example, communication between the device 101 and one or more external devices (e.g., sensors 102, an electronic device 104, or a server 106). For example, the communication interface 180 may communicate with the one or more external devices (e.g., the server 106 and/or the electronic device 104) by being connected to a network 162 through wireless communication or wired communication. The network 162 may include, for example, at least one of a telecommunications network, a computer network (e.g., LAN or WAN), the Internet, and/or a telephone network.

The communication interface 180 may be configured to communicate with the one or more external devices (e.g., sensors 102, or electronic device 104) via a wired communication interface 164, 165 or a wireless communication interface 164, 165. In an example, the wired communication may include, for example, at least one of Universal Serial Bus (USB), High Definition Multimedia Interface (HDMI), Recommended Standard-232 (RS-232), power-line communication, Plain Old Telephone Service (POTS), and the like. In an example, as a cellular communication protocol, the wireless communication interface 164, 165 may use at least one of Long-Term Evolution (LTE), LTE Advance (LTE-A), Code Division Multiple Access (CDMA), Wideband CDMA (WCDMA), Universal Mobile Telecommunications System (UMTS), Wireless Broadband (WiBro), Global System for Mobile Communications (GSM), and the like. In an example, the wireless communication interface 164, 165 may be configured to use a near-distance communication 164, 165. The near-distance communication interface 164, 165 may include for example, at least one of Wireless Fidelity (WiFi), Bluetooth, Bluetooth Low Energy (BLE), Near Field Communication (NFC), Global Navigation Satellite System (GNSS), and the like. According to a usage region or a bandwidth or the like, the GNSS may include, for example, at least one of Global Positioning System (GPS), Global Navigation Satellite System (Glonass), Beidou Navigation Satellite System (hereinafter, “Beidou”), Galileo, the European global satellite-based navigation system, and the like. Hereinafter, the “GPS” and the “GNSS” may be used interchangeably in the present document. In an example, the communication interface 180 may include or be communicably coupled to a transmitter, receiver and/or transceiver for communication with the external devices (e.g., sensors 102, or electronic device 104).

One or more sensors 102 may be in communication with the device via the communication interface connection 164. In an example, the device 101 and the sensors 102 may be configured as two separate devices or may be integrally combined into a signal device. As a single device the sensors 102 may be connected to the bus 110. For example, the sensors 102 may communicate with the one or more processors 120, the memory 140, the input/output interface 160, the display 170, and/or the communication interface 180 via the bus 110. The one or more sensors 102 may include one or more sensors capable of measuring electrodermal activity (EDA) of a user (e.g., subject). For example, the sensors 102 may be configured to measure the conductance associated with a surface of an area of skin of the user (e.g., subject). In an example, the sensors 102 may be integrated into a smart watch (e.g., device 101). In an example, the sensors 102 may be configured to output EDA data to the device 101 for further processing.

An electronic device 104 may be in communication with the device 101 via the communication interface connection 165. The electronic device 104 may include, for example, a head mounted display, a heads up display device, a laptop computer, a mobile phone, a smart phone, a tablet computer, and the like. In an example, the device 101 may be configured to receive the EDA data from the sensors 102 and output the EDA data to the electronic device 104 for further processing. For example, the electronic device 104 may be configured to generate the modified EDA signal based on the EDA data received from the device 101. The electronic device 104 may be configured to determine the health condition of the user (e.g., subject) based on the modified EDA signal. In an example, as a head mounted display or a heads up display device, the electronic device 104 may be configured to receive the physiological measurement (e.g., TVSymp) from the device 101 and output (e.g., display) the physiological measurement (e.g., TVSymp) or the indication associated with the health condition to the user (e.g., subject). For example, the electronic device 104 may output an alert, or notification, notifying the user of a possible impending seizure or other health conditions such as a risk of CNS-OT, or that the user (e.g., subject) may be experiencing symptoms of CNS-OT.

The server 106 may include a group of one or more servers. For example, all or some of the operations executed by the device 101 may be executed in a different one or a plurality of electronic devices (e.g., the device 101, the electronic device 104, and/or the server 106). In an example, if the device 101 needs to perform a certain function or service either automatically or based on a request, the device 101 may request at least some parts of functions related thereto alternatively or additionally to a different electronic device (e.g., the electronic device 104 and/or the server 106) instead of executing the function or the service autonomously. The different electronic devices (e.g., the electronic device 104, or the server 106) may execute the requested function or additional function, and may deliver a result thereof to the device 101. The device 101 may provide the requested function or service either directly or by additionally processing the received result. For example, a cloud computing, distributed computing, or client-server computing technique may be used. In an example, the server 106 may receive the indication associated with the health condition of the user (e.g., subject) and contact emergencies services based on the indication. In an example, the server may receive the physiological signal and/or the physiological measurement and store the physiological signal and/or the physiological measurement in one or more databases. The server 106 may group (e.g., in the one or more databases) the physiological signal and/or the physiological measurement based on the user (e.g., subject).

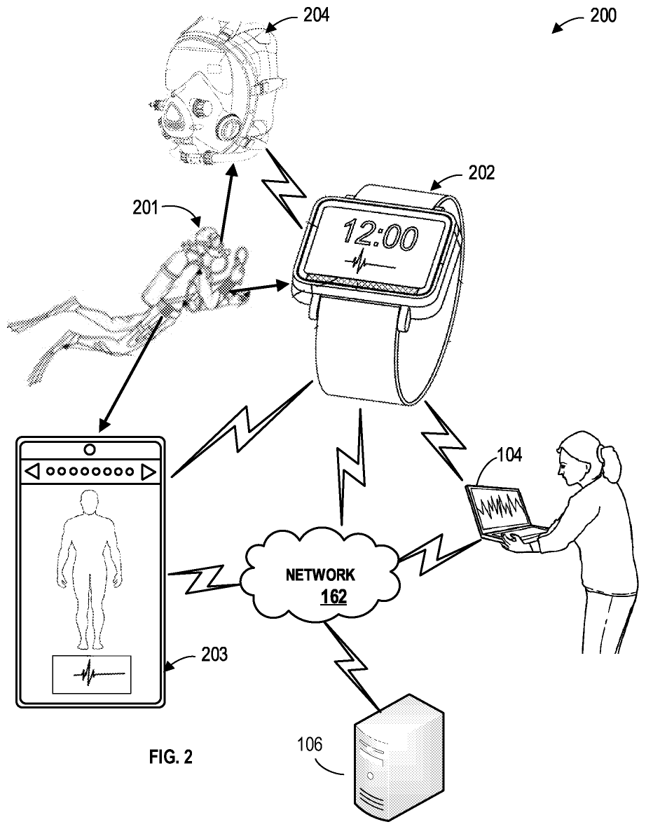

FIG. 2 shows an example system environment 200. The system may include a device 202 (e.g., smart watch, other wearable devices, and the like), a computing device 203 (e.g., smart phone, mobile phone, and the like), a display device 204 (e.g., head mounted display, heads up display device, and the like), the electronic device 104 (e.g., laptop computer, tablet computer, and the like), and/or a server 106. The device 202 may include the sensors 102. The sensors 102 may be affixed to the subject 201 such that the sensors 102 are in contact with a surface of skin of the subject 201. The sensors 102 may measure EDA of the subject and output the EDA to the computing device 203 for further processing. As shown in FIG. 2, the device 202 may be a fully integrated device configured to process the EDA measured by the sensors 102 and output an indication of a health condition based on the EDA to the subject 201. The health condition may include one or more of a risk of seizure, a risk of CNS-OT, or symptoms of CNS-OT. In an example, the computing device 203 may be configured to receive the EDA data from the device 202 and output the indication to the subject 201 based on the EDA data. In an example, the display device 204 may receive the indication from the device 202, and/or the computing device 203, and display the indication to the subject 201. In an example, the device 202 and/or the computing device 203 may be in communication with the electronic device 204 via network 162. For example the device 202 and/or the computing device 203 may output the EDA signal to the electronic device 104. For example, a separate user may use the electronic device 104 to monitor the EDA signal of the subject 201. The electronic device 104 may be configured to determine the health condition based on the EDA signal and output an indication of the health condition to the separate user via the electronic device 104. In an example, the device 202 and/or the computing device 203 may output the indication of the health condition to the electronic device 104 via network 162. The electronic device 104 may then output the received indication to the separate user. In an example, the device 202 and/or the computing device 203 may be in communication with the server via network 162. For example, the device 202 and/or the computing device 203 may output the EDA signal to the server 106. The server 106 may be configured to determine the health condition based on the EDA signal and contact emergency services based on the health condition. In an example, the device 202 and/or the computing device 203 may output the indication of the health condition to the server 106 via network 162. The server 106 may be configured to contact emergency services based on the received indication.

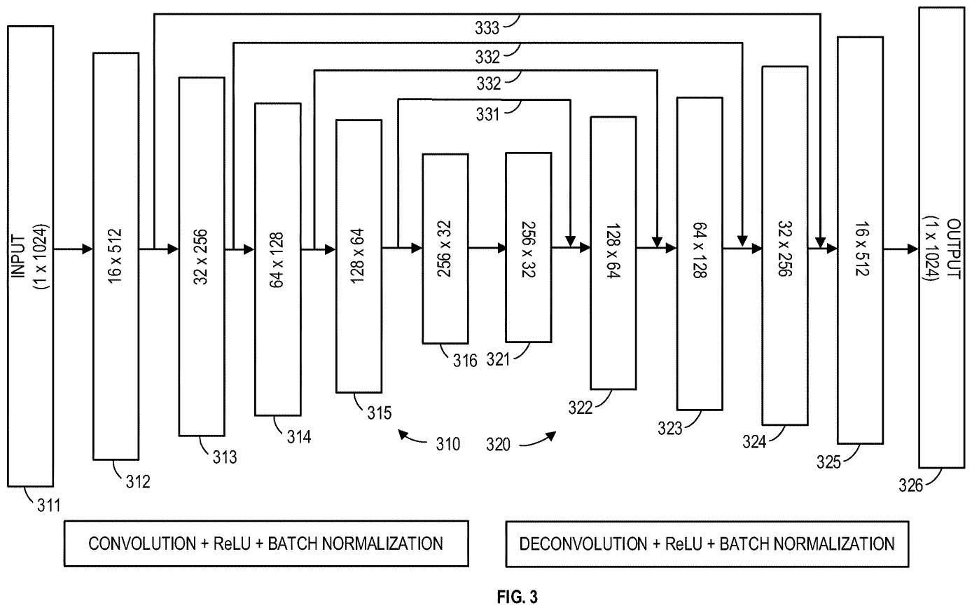

FIG. 3 shows an example machine learning system 300. For example, the machine learning system 300 may include a machine learning model such as a deep convolutional autoencoder (DCAE) network 300. The DCAE network 300 may be configured to include an encoder network 310 and a decoder network 320. The DCAE network 300 may be configured to encode data received at an input layer of the encoder network 310 through layers 312, 313, 314, and 315 to an output layer 316 of the encoder network 310. For example, input layer 311 may include a 1×1024 layer. Features may be upscaled through layers 321, 322, 323, 324, 325, to output layer 326 of decoder network 320. As an example, the decoder network 320 may mirror the encoder network 310 having the same quantity and size layers 321, 322, 323, 324, 325, 326 in reverse order. The DCAE network 300 may be trained to receive an EDA signal associated with a subject that may contain artifacts and output a modified EDA signal with reduced artifacts. For example, the artifacts may be caused by one or more of motion noise or electronic noise. For example, motion artifacts may be caused by variable conduction of signals as the subject moves about and electronic noise may be caused by from radiofrequency and magnetic interference.

As an example, the DCAE network 300 may include one or more convolutional blocks. Each convolutional block may include a 1D convolution layer (with stride=2), ReLU activation function, and batch normalization. Each convolutional block may reduce the input dimension by half. As shown in FIG. 3, the dimension of the data at every stage may include the number of features (e.g., channels)×time stamps. Convolution with stride may be used to reduce the dimension at each layer. The decoder network may be symmetric to the encoder network. The decoder network may deconvolve the encoded vector through different blocks. Each decoder block (except the last block which only consists of a transposed convolutional layer) may include a transposed convolution operation with stride 2 which may up sample the input sequence by a factor of 2, a ReLU activation function, and batch normalization. The DCAE network 300 may include skip connections (e.g., skip connections 331, 332, 333, 334) between one or more layers of the encoder network 310 and the decoder network 320. The skip connections (e.g., skip connections 331, 332, 333, 334) between encoder blocks and decoder blocks add the feature maps to the symmetric transposed convolution blocks. The skip connections (e.g., skip connections 331, 332, 333, 334) may transfer the signal details from the encoder network 310 to the decoder network 320, which may aid the recovery of the clean signal. For example, the skip connections (e.g., skip connections 331, 332, 333, 334) may be used to overcome the optimization difficulty caused by the vanishing gradient phenomena in deep learning architecture.

The mean squared error (MSE) may be used between the uncorrupted and the reconstructed signals, with L1 regularization on the model parameters as the loss criterion of the DCAE network 300. For example, this may prevent possible over-smoothing due to the MSE loss function. The loss function may be represented as

$$J = {\sum }_{i = 1}^{N} {\vert {x}_{i} - {\hat{y}}_{i} \vert}_{2}^{2} + λ {\vert W \vert}_{1} .$$

J may represent the loss criterion, which consists of MSE loss between the uncorrupted and the reconstructed signals with L1 regularization added by the parameter X. W may represent the DCA model 300 parameters and xᵢ and yᵢ may represent the original and the reconstructed signal for the i^{th} example. N may represent the total number of training samples.

One or more training data sets may be used to train the DCAE network 300. As an example, a training data set may include physiological signals from a public data set associated with physiological stimuli based on one or more of an auditory task, pain induced by electrical stimulation, a visual detection task, fear conditioning tasks, being shown aversive or neutral pictures, being shown pictures while subjected to auditory distractors, or being shown pictures of facial expressions. As an example, a training data set may include physiological signals associated with a prevalence of artifacts. As an example, a training data set may include physiological signals associated with one or more subjects experiencing central nervous system oxygen toxicity (CNS-OT) conditions. As an example, a training data set may include physiological signals associated with a first sub data set that may include physiological signals associated with a reduction of artifacts collected from both hands of one or more subjects and a second sub data set that may include physiological signals associated with a prevalence of artifacts collected from a first hand of one or more subjects and physiological signals associated with a reduction of artifacts collected from a second hand of one or more subjects.

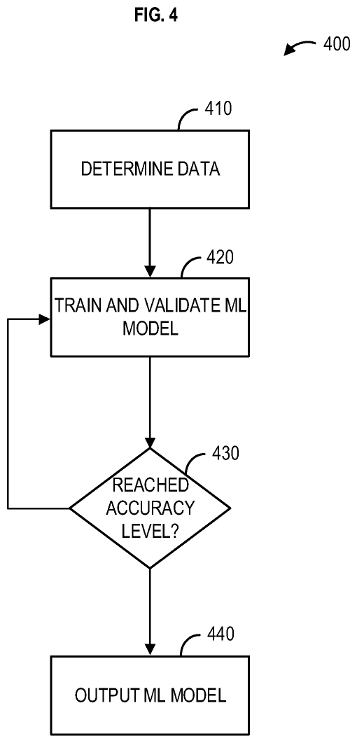

FIG. 4 shows a flowchart of an example training method 400 for generating the machine learning model (e.g., deep convolutional autoencoder (DCAE) network). The training 400 may be implemented by one or more user devices (e.g., the device 101, the electronic device 104, and/or the server 106). At step 410, one or more training data sets associated with one or more subjects may be determined (e.g., access, receive, retrieve, etc.). As an example, a training data set may include physiological signals from a public data set associated with physiological stimuli based on one or more of an auditory task, pain induced by electrical stimulation, a visual detection task, fear conditioning tasks, being shown aversive or neutral pictures, being shown pictures while subjected to auditory distractors, or being shown pictures of facial expressions. As an example, a training data set may include physiological signals associated with a prevalence of artifacts. As an example, a training data set may include physiological signals associated with one or more subjects experiencing central nervous system oxygen toxicity (CNS-OT) conditions. As an example, a training data set may include physiological signals associated with a first sub data set that may include physiological signals associated with a reduction of artifacts collected from both hands of one or more subjects and a second sub data set that may include physiological signals associated with a prevalence of artifacts collected from a first hand of one or more subjects and physiological signals associated with a reduction of artifacts collected from a second hand of one or more subjects.

At step 420, the machine learning model (e.g., DCAE network) may be trained based on the one or more training data sets. In an example, a first portion of the training data sets may be used to train the machine learning model (e.g., DCAE network). As an example, the DCAE may be trained and optimized by using an Adam optimizer with an initial learning rate of 0.001. The learning rate may be reduced by a factor of 0.1 after every five epochs. The training may end after 20 epochs.

At step 430, the machine learning model (e.g., DCAE network) may be evaluated using a second portion of the training data sets. For example, the machine learning model (e.g., DCAE network) may be evaluated to determine whether the predicted values have achieved a desired accuracy level. Once the desired accuracy level is achieved the machine learning model (e.g., DCAE network) may be output at step 440.

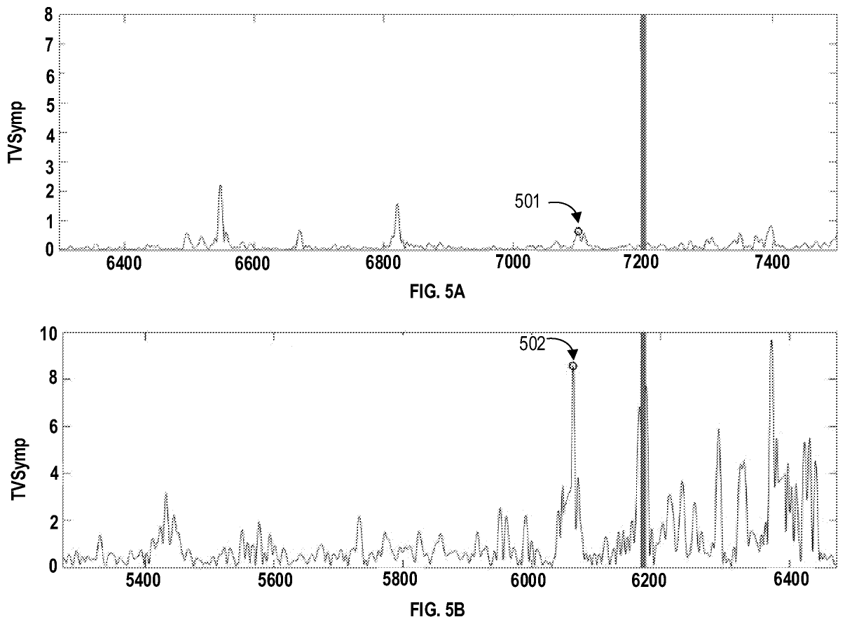

FIGS. 5A-5B show example physiological measurements (e.g., TVSymp) associated with a subject. For example, physiological data (e.g., EDA signal) associated with a subject may be used to generate a physiological measurement. The physiological measurement may include a time-invariant and a time-variant spectral analysis of the EDA signal (TVSymp). In an example, to compute the TVSymp, the time-frequency representation of EDA may be computed using variable frequency complex demodulation (VFCDM), which may enable a more accurate amplitude estimation along and may obtain increased time-frequency resolutions. At a sampling frequency of VFCDM decomposition at 2 Hz, the second and third components, including the approximate frequency range 0.08-0.25 Hz, may be used to compute TVSymp. Amplitudes of the time-varying components in this band at each time point may be summed together to obtain an estimated reconstructed EDA signal, X′(t). X′(t) may be normalized to a unit variance (making TVSymp a dimensionless quantity), and its instantaneous amplitude may be computed using the Hilbert transform, such as

$${Y}^{′} ( t ) = \frac{1}{π} P {\int }_{- ∞}^{∞} {X}^{′} ( τ ) / ( t - τ ) d τ .$$

P may indicate the Cauchy principal value. X′(t) and Y′(t) may form the complex conjugate pair. Thus, an analytic signal, Z(t), may be defined as Z(t)=X′(t)+iY′(t)=a(t)e^{jθ(t)}, where a(t)=[X′²(t)+Y′²(t)]^{1/2}, and θ(t)=arctan(Y′(t)/X′(t)). The resulting a(t) may be the instantaneous amplitude of Z(t) and may correspond to the TVSymp time series.

FIG. 5A shows an example physiological measurement (e.g., TVSymp) associated with a subject without CNS-OT exposure while FIG. 5B shows an example physiological measurement (e.g., TVSymp) associated with a subject exhibiting symptoms (e.g., diaphoresis) of CNS-OT. FIG. 5B shows a large increase in the physiological measurement (e.g., TVSymp) amplitude that may precede the occurrence of symptoms of CNS-OT. For example, as shown in FIG. 5B, the physiological measurement (e.g., TVSymp) may reach a value higher than 8, as shown by the circles 501, 502 in FIGS. 5A-5B. In an example, the physiological measurement (e.g., TVSymp) may show several prominent peaks even after the end of the HBO₂.

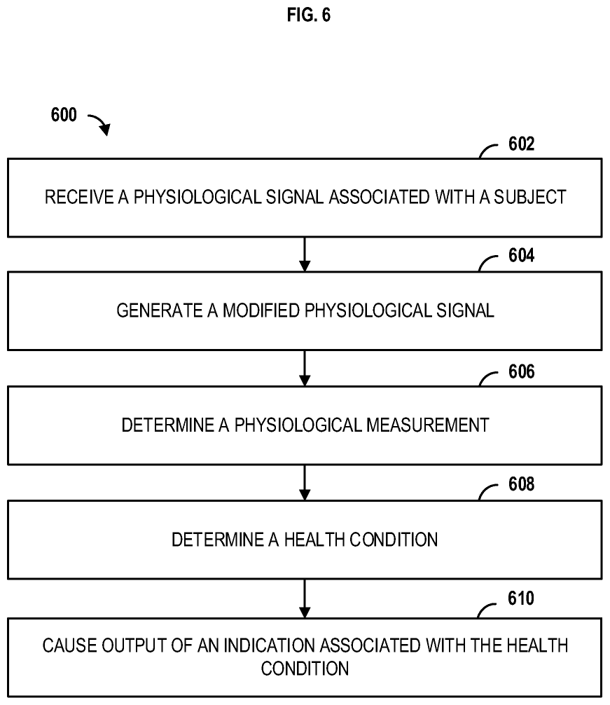

FIG. 6 shows a flowchart of an example method 600. Method 600 may be implemented by a user device (e.g., device 101, electronic device 104, sever 106). For example, the user device may include a smart watch, a smart phone, a laptop computer, a tablet computer, a desktop computer, a server, and the like. At step 602, a physiological signal associated with a subject may be received. For example, the physiological signal may be received from one or more sensors by the user device. The physiological signal may include an electrodermal activity (EDA) signal. The EDA signal may include artifacts. For example, the artifacts may be caused by one or more of motion noise or electronic noise. For example, motion artifacts may be caused by variable conduction of signals as the subject moves above and electronic noise may be caused by radiofrequency and magnetic interference.

At step 604, a modified physiological signal may be generated based on an application of a machine learning model to the physiological signal. For example, the user device may apply the machine learning model to the physiological signal to generate the modified physiological signal. The modified physiological signal may include the physiological signal with a reduction of the artifacts. The machine learning model may include a deep convolutional autoencoder (DCAE) network. The DCAE network may be trained based on one or more training data sets. As an example, a training data set may include physiological signals from a public data set associated with physiological stimuli based on one or more of an auditory task, pain induced by electrical stimulation, a visual detection task, fear conditioning tasks, being shown aversive or neutral pictures, being shown pictures while subjected to auditory distractors, or being shown pictures of facial expressions. As an example, a training data set may include physiological signals associated with a prevalence of artifacts. As an example, a training data set may include physiological signals associated with one or more subjects experiencing central nervous system oxygen toxicity (CNS-OT) conditions. As an example, a training data set may include physiological signals associated with a first sub data set that may include physiological signals associated with a reduction of artifacts collected from both hands of one or more subjects and a second sub data set that may include physiological signals associated with a prevalence of artifacts collected from a first hand of one or more subjects and physiological signals associated with a reduction of artifacts collected from a second hand of one or more subjects.

At step 606, a physiological measurement may be determined based on the modified physiological signal. For example, the user device may determine the physiological measurement based on the modified physiological signal. The physiological measurement may include a time-invariant and/or a time-variant spectral analysis of the EDA signal (TVSymp).

At step 608, a health condition may be determined based on a change in the physiological measurement satisfying a threshold. For example, the user device may determine the health condition based on the change in the physiological measurement satisfying a threshold. The change may be based on an increase in phasic components of the EDA signal. For example, the change may be caused by stress experienced by the subject based on breathing performed by the subject during prolonged exposure to HBO₂. The health condition may include one or more of a risk of seizure, a risk of CNS-OT, or symptoms of CNS-OT. In an example, it may be first determined that the physiological measurement satisfies a condition. The condition may include one or more of an autonomic induced elevation of a phasic component of the EDA signal or a non-autonomic induced elevation of a phasic component of the EDA signal. The change in the physiological measurement may be determined to satisfy the threshold based on the physiological measurement satisfying the condition.

At step 610, an indication associated with the health condition may be output. For example, the user device may output the health condition to the subject via a display (e.g., interface) of the user device. For example, the indication may include an alert notifying the user (e.g., subject) of a possible impending seizure or other health conditions such as a risk of CNS-OT, or that the user (e.g., subject) may be experiencing symptoms of CNS-OT.

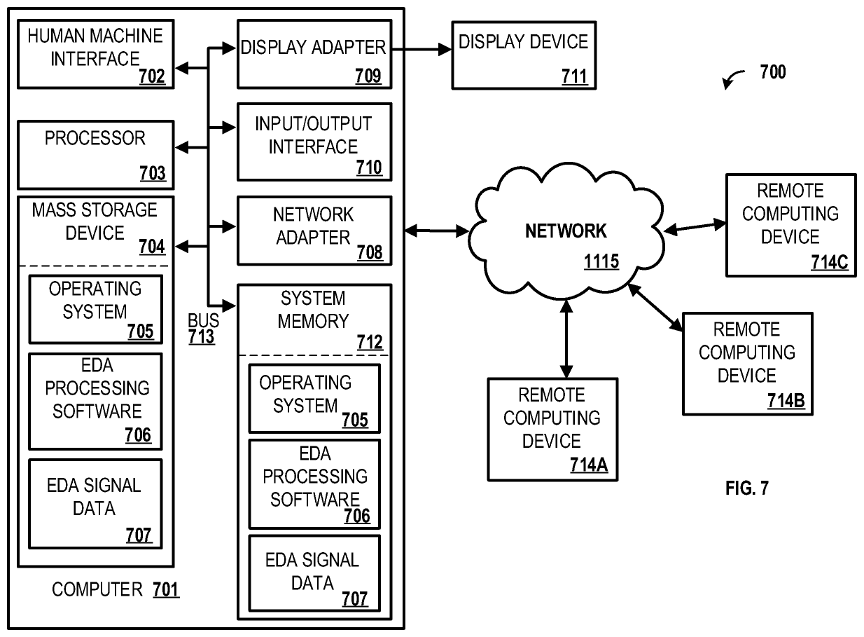

In an example, the methods and systems may be implemented on a computer 701 as shown in FIG. 7 and described below. By way of example, device 101, electronic device 104, and server 106 of FIG. 1 may be a computer 701 as shown in FIG. 7. Similarly, the methods and systems disclosed can utilize one or more computers to perform one or more functions in one or more locations. FIG. 7 shows a block diagram of an example operating environment 700 for performing the disclosed methods. For example, operating environment 700 is only an example of an operating environment and is not intended to suggest any limitation as to the scope of use or functionality of operating environment architecture. Neither should the operating environment 700 be interpreted as having any dependency or requirement relating to any one or combination of components illustrated in the example operating environment 700.

The present methods and systems can be operational with numerous other general purpose or special purpose computing system environments or configurations. Examples of well-known computing systems, environments, and/or configurations that can be suitable for use with the systems and methods include, but are not limited to, personal computers, server computers, laptop devices, and multiprocessor systems. Additional examples include set top boxes, programmable consumer electronics, network PCs, minicomputers, mainframe computers, distributed computing environments that include any of the above systems or devices, and the like.

The processing of the disclosed methods and systems can be performed by software components. The disclosed systems and methods can be described in the general context of computer-executable instructions, such as program modules, being executed by one or more computers or other devices. Generally, program modules include computer code, routines, programs, objects, components, data structures, and/or the like that perform particular tasks or implement particular abstract data types. The disclosed methods can also be practiced in grid-based and distributed computing environments where tasks are performed by remote processing devices that are linked through a communications network. In a distributed computing environment, program modules can be located in local and/or remote computer storage media such as memory storage devices.

Further, one skilled in the art will appreciate that the systems and methods disclosed herein can be implemented via a general-purpose computing device in the form of a computer 701. The computer 701 can include one or more components, such as one or more processors 703, a system memory 712, and a bus 713 that couples various components of the computer 701 comprising the one or more processors 703 to the system memory 712. In the case of multiple processors 703, the computer 701 may utilize parallel computing.

The bus 713 may include one or more of several possible types of bus structures, such as a memory bus, memory controller, a peripheral bus, an accelerated graphics port, or local bus using any of a variety of bus architectures. By way of example, such architectures can include an Industry Standard Architecture (ISA) bus, a Micro Channel Architecture (MCA) bus, an Enhanced ISA (EISA) bus, a Video Electronics Standards Association (VESA) local bus, an Accelerated Graphics Port (AGP) bus, and a Peripheral Component Interconnects (PCI), a PCI-Express bus, a Personal Computer Memory Card Industry Association (PCMCIA), Universal Serial Bus (USB) and the like. The bus 713, and all buses specified in this description can also be implemented over a wired or wireless network connection and one or more of the components of the computer 701, such as the one or more processors 703, a mass storage device 704, an operating system 705, EDA processing software 706, EDA signal data 707, a network adapter 708, the system memory 712, an Input/Output Interface 710, a display adapter 709, a display device 711, and a human machine interface 702, can be contained within one or more remote computing devices 714A-714C at physically separate locations, connected through buses of this form, in effect implementing a fully distributed system.

The computer 701 may operate on and/or include a variety of computer-readable media (e.g., non-transitory). Computer-readable media may be any available media that is accessible by the computer 701 and includes non-transitory, volatile, and/or non-volatile media, removable and non-removable media. The system memory 712 has computer-readable media in the form of volatile memory, such as random access memory (RAM), and/or non-volatile memory, such as read-only memory (ROM). The system memory 712 typically can include data such as the EDA signal data 707 and/or program modules such as the operating system 705 and the EDA processing software 706 that are accessible to and/or are operated on by the one or more processors 703.

The computer 701 may also include other removable/non-removable, volatile/non-volatile computer storage media. The mass storage device 704 may provide non-volatile storage of computer code, computer-readable instructions, data structures, program modules, and other data for the computer 701. The mass storage device 704 may be a hard disk, a removable magnetic disk, a removable optical disk, magnetic cassettes or other magnetic storage devices, flash memory cards, CD-ROM, digital versatile disks (DVD) or other optical storage, random access memories (RAM), read-only memories (ROM), electrically erasable programmable read-only memory (EEPROM), and the like.

Any number of program modules can be stored on the mass storage device 704, such as, by way of example, the operating system 705 and the EDA processing software 706. One or more of the operating system 705 and the EDA processing software 706 (or some combination thereof) can include elements of the programming and the EDA processing software 706. The EDA signal data 707 can also be stored on the mass storage device 704. The EDA signal data 707 can be stored in any of one or more databases known in the art. Examples of such databases include, DB2®, Microsoft® Access, Microsoft® SQL Server, Oracle®, mySQL, PostgreSQL, and the like. The databases can be centralized or distributed across multiple locations within the network 715.

A user may enter commands and information into the computer 701 via an input device (not shown). Such input devices may include, but are not limited to, a keyboard, pointing device (e.g., a computer mouse, remote control), a microphone, a joystick, a scanner, tactile input devices such as gloves, and other body coverings, motion sensor, and the like These and other input devices may be connected to the one or more processors 703 via a human-machine interface 702 that is coupled to the bus 713, but may be connected by other interface and bus structures, such as a parallel port, game port, an IEEE 1394 Port (also known as a Firewire port), a serial port, network adapter 708, and/or a universal serial bus (USB).

A display device 711 may also be connected to the bus 713 via an interface, such as a display adapter 709. It is contemplated that the computer 701 may have more than one display adapter 709 and the computer 701 may have more than one display device 711. A display device 711 may be a monitor, an LCD (Liquid Crystal Display), light-emitting diode (LED) display, television, smart lens, smart glass, and/or a projector. In addition to the display device 711, other output peripheral devices may include components such as speakers (not shown) and a printer (not shown) which may be connected to the computer 701 via Input/Output Interface 710. Any step and/or result of the methods may be output (or caused to be output) in any form to an output device. Such output may be any form of visual representation, including, but not limited to, textual, graphical, animation, audio, tactile, and the like. The display 711 and computer 701 may be part of one device, or separate devices.

The computer 701 can operate in a networked environment using logical connections to one or more remote computing devices 714A-714C. By way of example, a remote computing device 714A-714C can be a personal computer, computing station (e.g., workstation), portable computer (e.g., laptop, mobile phone, tablet device), smart device (e.g., smartphone, smart watch, activity tracker, smart apparel, smart accessory), security and/or monitoring device, a server, a router, a network computer, a peer device, edge device or other common network node, and so on. Logical connections between the computer 701 and a remote computing device 714A-714C can be made via a network 715, such as a local area network (LAN) and/or a general wide area network (WAN). Such network connections can be through the network adapter 708. The network adapter 708 can be implemented in both wired and wireless environments. Such networking environments are conventional and commonplace in dwellings, offices, enterprise-wide computer networks, intranets, and the Internet.

Application programs and other executable program components such as the operating system 705 are illustrated herein as discrete blocks, although it is recognized that such programs and components can reside at various times in different storage components of the computing device 701, and are executed by the one or more processors 703 of the computer 701. An implementation of the EDA processing software 706 can be stored on or transmitted across some form of computer readable media. Any of the disclosed methods can be performed by computer readable instructions embodied on computer readable media. Computer readable media can be any available media that can be accessed by a computer. By way of example and not meant to be limiting, computer readable media can include “computer storage media” and “communications media.” “Computer storage media” can include volatile and non-volatile, removable and non-removable media implemented in any methods or technology for storage of information such as computer readable instructions, data structures, program modules, or other data. For example, computer storage media may include RAM, ROM, EEPROM, flash memory or other memory technology, CD-ROM, digital versatile disks (DVD) or other optical storage, magnetic cassettes, magnetic tape, magnetic disk storage or other magnetic storage devices, or any other medium which can be used to store the desired information and which can be accessed by a computer.

Each of the elements described in the present disclosure may include one or more components, and names thereof may vary depending on a type of an electronic device. The electronic device according to various exemplary embodiments may include at least one of the elements described in the present disclosure. Some of the elements described herein may be omitted and/or additional other elements may be further included. Further, some of the elements of the electronic device, according to various exemplary embodiments, may be combined and constructed as a single entity, so as to equally perform functions of the corresponding elements before combination.

It is to be understood that the methods and systems are not limited to specific methods, specific components, or to particular implementations. It is also to be understood that the terminology used herein is for the purpose of describing particular embodiments only and is not intended to be limiting.

As used in the specification and the appended claims, the singular forms “a,” “an” and “the” include plural referents unless the context clearly dictates otherwise.

Throughout the description and claims of this specification, the words “include” and/or “comprise” and variations of the words, such as “including,” “includes,” “comprising,” and “comprises,” mean “including but not limited to,” and is not intended to exclude, for example, other components, integers or steps. “Exemplary” means “an example of” and is not intended to convey an indication of a preferred or ideal embodiment. “Such as” is not used in a restrictive sense, but for explanatory purposes.

Disclosed are components (e.g., apparatuses) that can be used to perform the disclosed methods and can be part of the disclosed systems. These and other components are disclosed herein, and it is understood that when combinations, subsets, interactions, groups, etc. of these components are disclosed that while specific reference of each various individual and collective combinations and permutations of these may not be explicitly disclosed, each is specifically contemplated and described herein, for all methods and systems. This applies to all aspects of this application including, but not limited to, steps in disclosed methods. Thus, if there are a variety of additional steps that can be performed it is understood that each of these additional steps can be performed with any specific embodiment or combination of embodiments of the disclosed methods.

As will be appreciated by one skilled in the art, the methods and systems may take the form of an entirely hardware embodiment, an entirely software embodiment, or an embodiment combining software and hardware aspects. Furthermore, the methods and systems may take the form of a computer program product on a computer-readable storage medium (e.g., non-transitory) having processor-executable instructions (e.g., computer software) embodied in the storage medium. More particularly, the present methods and systems may take the form of web-implemented computer software. Any suitable computer-readable storage medium may be utilized including hard disks, CD-ROMs, optical storage devices, magnetic storage devices, memresistors, Non-Volatile Random Access Memory (NVRAM), flash memory, or a combination thereof.

Some additional examples of machine learning concepts and techniques that may be useful with the teachings herein include, for example, Supervised Machine Learning models, such as: “Logistic Regression” which may be used to determine if an input belongs to a certain group or not; “Support Vector Machines” which may be used to create coordinates for each object in an n-dimensional space and uses a hyperplane to group objects by common features; “Naive Bayes” which is an algorithm that may assume independence among variables and use probability to classify objects based on features; “Decision trees” which are also classifiers that may be used to determine what category an input falls into by traversing the leaf's and nodes of a tree; “Linear Regression” which may be used to identify relationships between the variable of interest and the inputs, and predict its values based on the values of the input variables; “k Nearest Neighbors” technique which involves grouping the closest objects in a dataset and finding the most frequent or average characteristics among the objects; “Random Forest” which is a collection of many decision trees from random subsets of the data, resulting in a combination of trees that may be more accurate in prediction than a single decision tree; “Boosting Algorithms”, such as Gradient Boosting Machine, XGBoost, and LightGBM, which use ensemble learning to combine predictions from multiple algorithms (such as decision trees) while taking into account the error from the previous algorithm. Additionally, Unsupervised Machine Learning models such as “K-Means” which is an algorithm that finds similarities between objects and groups them into K different clusters; and “Hierarchical Clustering” which build a tree of nested clusters without having to specify the number of clusters, can provide some functionality.

In some embodiments, the device 101 may implement a bounded edge-processing architecture configured to perform artifact reduction, physiological feature extraction, and health-condition prediction using deterministic computational resources associated with a wearable device carried by a diver. The bounded edge-processing architecture may limit processor utilization, memory utilization, communication bandwidth utilization, and processing latency associated with electrodermal activity (EDA) processing operations. In some embodiments, the bounded edge-processing architecture may operate without transmitting raw physiological signals to a remote cloud computing system. The bounded edge-processing architecture bounds or limits certain computational functions to ensure that an output is provided in sufficient time to provide a timely warning to a diver or an action that prevents the diver from experience oxygen toxicity.

In some embodiments, the device 101 may implement bounded real-time processing constraints associated with underwater physiological monitoring. For example, the device 101 may process EDA signal segments within a predetermined processing interval associated with a sliding acquisition window. In some embodiments, the predetermined processing interval may be less than 500 milliseconds, less than 250 milliseconds, or less than 100 milliseconds following acquisition of the EDA signal segment from the sensor 102. As with the bounded edge-processing architecture, the bounded real-time processing constraints provide an output in sufficient time to provide a timely warning to a diver or an action that prevents the diver from experiencing oxygen toxicity (CNS-OT).

In some embodiments, the artifact reduction program 157 may implement motion-aware adaptive filtering operations configured to distinguish autonomic-induced EDA responses from non-autonomic motion artifacts. Motion-aware adaptive filtering operations may include determining one or more motion vectors (e.g. motion amplitude and direction) based on accelerometer signals, gyroscope signals, pressure signals, or inertial measurement unit (IMU) signals associated with movement of the subject. The motion vectors may be temporally aligned with the EDA signal to identify artifact-corrupted intervals. Temporal alignment may be provided by a time reference such as a time stamp or other synchronizing signal.

In some embodiments, the artifact reduction program 157 may generate a signal-quality metric associated with the EDA signal. The signal-quality metric may be determined based on one or more of: electrode impedance measurements, motion magnitude measurements, saturation measurements, clipping measurements, spectral discontinuity measurements, baseline instability measurements, or sensor-detachment determinations. In some embodiments, the signal-quality metric may be compared against a signal-quality threshold to determine whether physiological processing operations are permitted for a corresponding EDA signal segment. The signal-quality threshold may be obtained by experience with a cohort of divers having similar experience, by experience with the specific diver of interest being monitored, and/or by analysis.

In some embodiments, the artifact reduction program 157 may selectively gate physiological processing operations based on the signal-quality metric. For example, physiological measurements may be suppressed, delayed, recalculated, weighted, or discarded responsive to the signal-quality metric failing to satisfy the signal-quality threshold. The gating operations may reduce false-positive CNS-OT determinations arising from motion artifacts, underwater pressure changes, intermittent electrode contact, or non-physiological signal distortions. The artifact reduction program 157 can increase the accuracy of predicting oxygen toxicity by not processing signal-degrading artifacts using the gating operation.

In some embodiments, the machine learning model may comprise a bounded convolutional autoencoder network configured for execution on a wearable processor having constrained computational resources. The bounded convolutional autoencoder network may comprise a reduced parameter set, quantized weights, compressed activation layers, sparsified (i.e., made less dense) convolutional kernels, or fixed-point arithmetic operations configured to reduce computational complexity while maintaining artifact suppression accuracy. The bounded convolutional autoencoder network is bounded as with other computational to aid in providing an output in sufficient time to provide a timely warning to a diver or an action that prevents the diver from experiencing oxygen toxicity.

In some embodiments, the machine learning model may receive synchronized multi-modal physiological inputs comprising EDA signals and one or more of: accelerometer signals, gyroscope signals, pulse oximetry signals, heart rate signals, respiration signals, skin temperature signals, ambient pressure signals, depth measurements, oxygen partial pressure measurements, or diver breathing-cycle measurements. The machine learning model can be trained with these same signals that would be received from a diver or individual not being subject to events leading to oxygen-toxicity (i.e., normal signals. Hence, signals that deviate from the normal signals can indicate the onslaught of oxygen-toxicity.

In some embodiments, the machine learning model may generate a modified EDA signal using a constrained inference pipeline configured to satisfy a deterministic execution interval. The constrained inference pipeline may execute according to a predetermined scheduling interval such that each inference operation completes within a bounded processing duration associated with underwater diver monitoring to ensure the diver gets enough time to terminate the breathing of high concentrations of oxygen to avoid oxygen toxicity or to take other preventive actions.

In some embodiments, the signal processing program 159 may determine a time-variant sympathetic index based on overlapping spectral windows associated with the modified EDA signal. In some embodiments, the overlapping spectral windows may be generated using a bounded sliding-window operation configured to reduce memory utilization associated with storage of physiological data. Reduced memory requirements can translate to reduced hardware requirements so that necessary hardware is of a size that can be conveniently carried by a diver underwater.

In some embodiments, the device 101 may generate a CNS-OT risk state based on a combination of: the signal-quality metric, a motion-state determination, a spectral sympathetic index, a breathing-gas exposure duration, an oxygen partial pressure measurement, and a physiological trend associated with the subject. In some embodiments, the CNS-OT risk state may comprise a multi-level risk classification associated with escalating diver intervention operations. For example, an indication of a slower onslaught of oxygen toxicity may lead to reducing oxygen intake at a lower reduced level than an indication of a much quicker onslaught of oxygen toxicity that would require a much more reduced level of oxygen intake or other immediate preventive actions.

In some embodiments, responsive to determining the CNS-OT risk state satisfies an intervention threshold, the device 101 may automatically initiate one or more intervention operations. The intervention operations may include generating a tactile alert, generating an audible alert, activating a visual alert, causing modification of a breathing-gas delivery operation, causing activation of a diver ascent-assistance operation, causing transmission of an emergency communication, or causing activation of a decompression-control operation. Causing modification of a breathing-gas delivery operation or causing activation of a decompression-control operation relate to manually or automatically reducing oxygen intake of a diver or subject of interest from a diving gas supply system.

In some embodiments, the device 101 may maintain a rolling physiological buffer storing a bounded quantity of physiological data associated with the subject. The rolling physiological buffer may overwrite older physiological data responsive to storage utilization satisfying a storage threshold. The rolling physiological buffer may thereby reduce memory utilization and power consumption associated with long-duration underwater monitoring. Reducing memory utilization and power consumption can translate to reduced hardware requirements so that necessary hardware is of a size that can be conveniently carried by a diver underwater.

In some embodiments, the device 101 may operate in a disconnected operational mode in which physiological processing operations continue during interruption of wireless communication with a remote computing system. In some embodiments, the device 101 may continue determining CNS-OT risk states and generating diver intervention operations during the disconnected operational mode to provide continuous diver safety monitoring. That is, the device 101 is configured to operate autonomously without the need to communicate with a remote computing system (i.e., a computing system not located with the diver).

In some embodiments, the device 101 may implement adaptive sampling-rate control associated with physiological data acquisition. For example, the device 101 may increase a sampling rate responsive to detection of elevated sympathetic activity and may decrease the sampling rate responsive to determination of a stable physiological state. The adaptive sampling-rate control may reduce power utilization associated with wearable underwater operation. Reduce power utilization can translate to reduced battery requirements so that necessary hardware is of a size that can be conveniently carried by a diver underwater.

In some embodiments, the device 101 may implement hardware-level synchronization between EDA acquisition circuitry and motion-sensing circuitry such that motion-related artifacts are identified within a bounded temporal alignment interval. The hardware-level synchronization may reduce timing drift between independently acquired physiological and motion signals. Reducing timing drift between independently acquired physiological and motion signals can provide a more accurate prediction of a CNS-OT risk state.

In some embodiments, the device 101 may implement an adaptive artifact suppression profile associated with a particular diver, dive environment, or breathing-gas exposure condition. The adaptive artifact suppression profile may be updated responsive to accumulated physiological data associated with prior dives performed by the subject. The adaptive artifact suppression profile that is being updated can provide a more accurate prediction of a CNS-OT risk state.

In some embodiments, the device 101 may implement a closed-loop physiological safety system configured to continuously acquire physiological measurements, reduce motion artifacts using bounded edge processing, determine a CNS-OT risk state, and automatically initiate diver intervention operations responsive to the CNS-OT risk state satisfying an intervention threshold. Diver intervention operations can include manually or automatically reducing oxygen intake of a diver or subject of interest to reduce the probability of CNS-OT occurring the diver. For example, a controller receiving indication of a high CNS-OT risk state can implement an algorithm to control an amount of oxygen in a breathing loop of a rebreather or breathing gas supply system carried by a diver in a feedback control loop. In another example, diver intervention operations can include dispatching safety divers to aid the diver having the high CNS-OT risk state upon receipt of an indication of the high CNS-OT risk state.

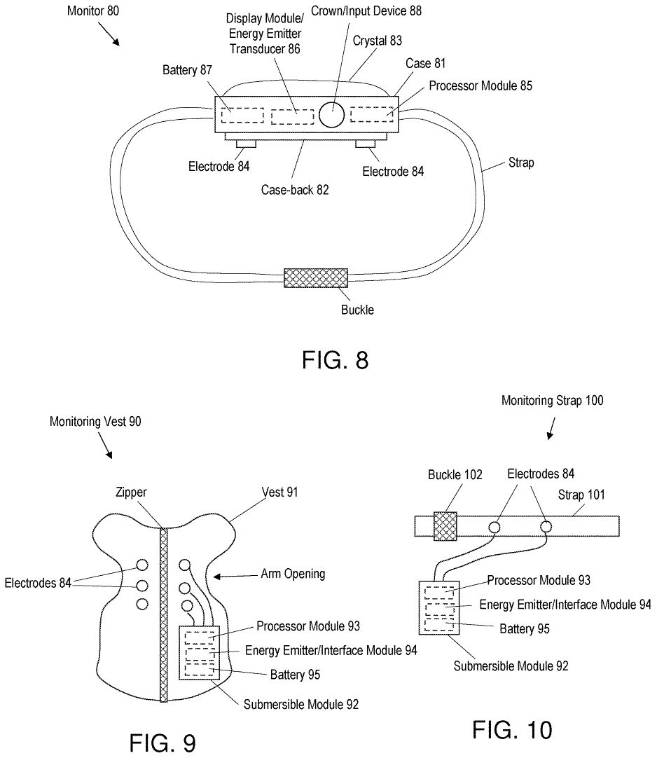

Discussed next are various embodiments of hardware used for implementing the disclosure herein. FIG. 8 illustrates a side view of a wrist-worn monitor 80. The monitor 80 is shaped and configured similar to a dive watch. The monitor 80 may use dive watch technology to keep the monitor 80 waterproof and pressure proof. The monitor 80 may include a time module (not shown) for displaying time to a diver. The monitor 80 includes case 81 and a case-back 82 providing access to the interior of the monitor 80. The case and case-back may be made of a corrosion-resistant material such as stainless steel and/or titanium. The monitor 80 also includes a pressure-resistant crystal 83 to provide visual access into an interior of monitor 80. In one or more embodiments, the crystal 83 is made of sapphire. The monitor 80 further includes one of more electrodes 84 secured to the case-back 82 and configured to contact a wrist of the diver to collect EDA data from the diver.

The electrodes 84 may be made of a corrosion-resistant conductive material such as stainless steel and/or titanium. The monitor 80 may include an array of electrodes 84 to collect EDA data at multiple locations on the wrist so that the EDA data from the multiple locations can be compared for quality-check purposes.

Disposed inside the monitor 80 is a processor module 85. The processor module 85 includes a processor and memory to receive and process the EDA data to determine CNS-OT risk in accordance with the techniques disclosed herein. Also disposed inside the monitor 80 is a display module/energy emitter transducer 86. The display module/energy emitter transducer 86 is configured to display information related to operation of the monitor 80. For example, the display module can display the determined CNS-OT risk either binary (e.g., either risk on or risk off) or according to a spectrum of risk. The display module can also an alert signal to inform the diver that CNS-OT is predicted. The alert signal can include a flashing display. The energy emitter transducer is configured to emit energy such as acoustic energy or vibration energy to alert the diver that CNS-OT is predicted. Also disposed inside the monitor 80 is a battery 87 configured to provide power the processor module 85 and the display module/energy emitter transducer 86. The battery 87 can be a rechargeable battery that can be charged by induction or by solar energy with a face of the monitor 80 having a solar cell.

The monitor 80 can also include an input device 88 such as for turning the monitor 80 on and off and/or inputting information into the monitor 80 such as for scrolling through a menu and inputting or extracting selected information. The input device 88 can be a pushbutton or a watch-like crown. To keep the monitor 80 secured to a wrist, the monitor 80 includes a strap with a buckle.

FIG. 9 illustrates a monitoring vest 90. The monitoring vest 90 includes a vest 91 configured to be worn by a diver or subject. The vest 91 can be worn under an external garment such as a wet suit or dry suit. The vest 91 can be made of an elastic material such as neoprene and to fit snugly to keep the electrodes 84 in contact with the body of the diver or subject. The electrodes 84 are in communication, such as by wires for example, with a submersible module 92. The submersible module 92 is configured to be water and pressure tight and can be made of a corrosion resistant material such as stainless steel, aluminum, or a composite material. The submersible module 92 includes a processor module 93, an energy emitter/interface module 94, and a battery 95. The processor module 93 includes a processor and memory to receive and process the EDA data to determine CNS-OT risk in accordance with the techniques disclosed herein. The energy emitter/interface module 94 is configured to emit energy such as acoustic energy or vibration energy to alert the diver to impending CNS-OT. In one or more embodiments, the acoustic energy can be emitted at different tones with each tone corresponding to a level of CNS-OT risk. In one example, the tone can be pulsed at the highest level of risk. In addition, a separate tone can be emitted periodically to let the diver know that the monitoring vest 90 is functioning correctly. The battery 95 is configured to supply power to the submersible module 92. The battery 95 can be user replaceable and/or rechargeable. The submersible module 92 can also include an input device (not shown) similar to the input device 88 for turning the monitoring vest 90 on or off and inputting various types of information as needed using a local display (also not shown).

FIG. 10 illustrates a monitoring strap 100. The monitoring strap 100 is similar to the monitoring vest 90 and has similar components but uses a strap 101 and buckle 102 to secure the electrodes 84 to the body of the diver or subject rather than a vest. The strap 101 can be secured a chest of the diver or a wrist of the diver.

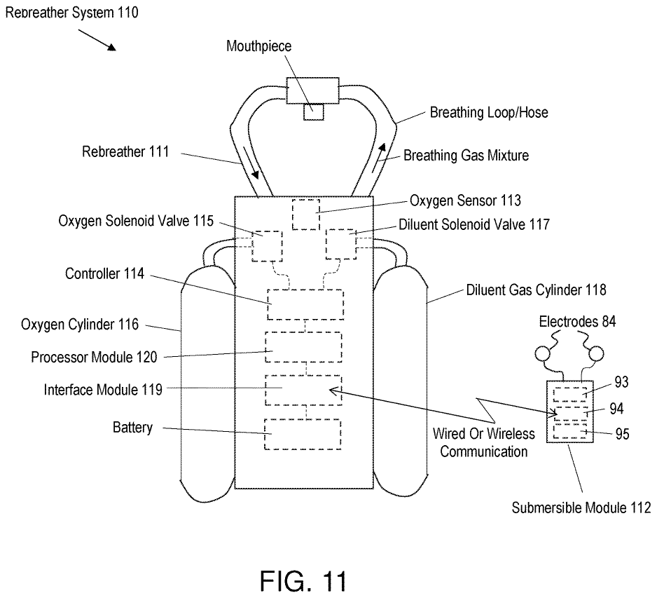

FIG. 11 illustrates a rebreather system 110. The rebreather system 110 includes a rebreather 111 and a submersible module 112. The rebreather 111 is configured provide breathing gas to a diver where the gas has a selected normal oxygen concentration corresponding to the depth of the diver. To provide the breathing gas at a selected oxygen concentration, the rebreather 111 is configured to recycle the gas exhaled by the diver. Recycling the exhaled gas includes removing or scrubbing carbon dioxide from the exhaled gas and adding oxygen and a diluent gas such as air as needed to provide breathing gas at the selected concentration of oxygen. The rebreather 111 includes an oxygen sensor 113 configured to sense a concentration of oxygen in a breathing loop from which the diver will breathe. The oxygen sensor 113 provides input to a controller 114. The controller 114 is configured to open an oxygen solenoid valve 115 to admit oxygen from an oxygen cylinder 116 to the breathing loop. The controller 114 is also configured to open a diluent solenoid valve 117 to admit diluent gas from a diluent gas cylinder 118 to the breathing loop. Thus, the controller 114 can control the concentration of oxygen in the breathing loop at a setpoint using feedback from the oxygen sensor 113. The controller 114 can be configured to implement various algorithms such as set point analog control (e.g., proportional-integral-derivative control), a lookup table based on analysis or testing, model-based control wherein individual components and their interactions are mathematically modeled based on physics, and/or machine-learning in which a neural network is trained using training data based on various scenarios.

The rebreather 111 includes an interface module 119 configured to receive data from the submersible module 112. The data may be transmitted using a wired system or wirelessly such as by using complementary acoustic modems. EDA data is obtained by submersible module 112 using the electrodes 84. The CNS-OT risk can be determined by the processor module 85 and transmitted to the interface module 119. Alternatively, raw EDA data can be transmitted from the submersible module 112 to the interface module 119. A processor module 120 in the rebreather 111 can then determine the CNS-OT risk. The CNS-OT risk no matter how determined is used by the processor module 120 to determine an appropriate oxygen concentration setpoint that is transmitted to the controller 114. In one or more embodiments, the CNS-OT risk is determined on a binary process in which there in no risk or positive risk such as whether the risk is below a risk threshold (i.e., no risk) or above the risk threshold (i.e., positive risk). Based on determining positive risk, the controller 114 immediately lowers the oxygen concentration setpoint to a minimum level that will still support life at a depth of the diver. Accordingly, a pressure sensor (not shown) may be used to provide depth input to the processor module 120. Upon CNS-OT risk being lowered to an acceptable level as determined by using data from the electrodes 84, the oxygen concentration setpoint can be raised to the normal setpoint. Thus, a closed-loop control system is established starting with obtaining EDA data using the electrodes 84, then determining the corresponding CNS-OT risk, then lowering the oxygen concentration setpoint if the risk is determined to be unacceptable, and finally determining if the lowered oxygen concentration setpoint has resulted in lowering the CNS-OT risk based on sensed EDA data. In this manner, the oxygen concentration setpoint in the rebreather 111 can be lowered or raised accordingly to ensure the safety of the diver. The rebreather 111 includes a battery 121 for powering the various modules and equipment requiring electrical power in the rebreather 111. The battery 121 can be rechargeable and/or user replaceable.

Alternatively, in one or more embodiments, a spectrum of CNS-OT risks may be determined and the controller 114 can set the oxygen concentration setpoint based on a level of the CNS-OT risk such that for a low level of risk the setpoint is not lowered as much as the setpoint would be lowered for a higher level of risk.

In another embodiment, a diver may be undergoing treatment for decompression sickness in a recompression chamber. Part of the therapy generally includes breathing increased partial pressure of oxygen at pressure greater than atmospheric pressure (i.e., breathing hyperbaric oxygen). The breathing gas is supplied by a breathing gas supply system. Breathing hyperbaric oxygen, however, can lead to CNS-OT in some cases. Hence, monitoring the patient in the recompression chamber using the device 101 and reducing a partial pressure of oxygen being breathed either manually or automatically in response to receiving the alert signal can provide an extra level of patient wellbeing.

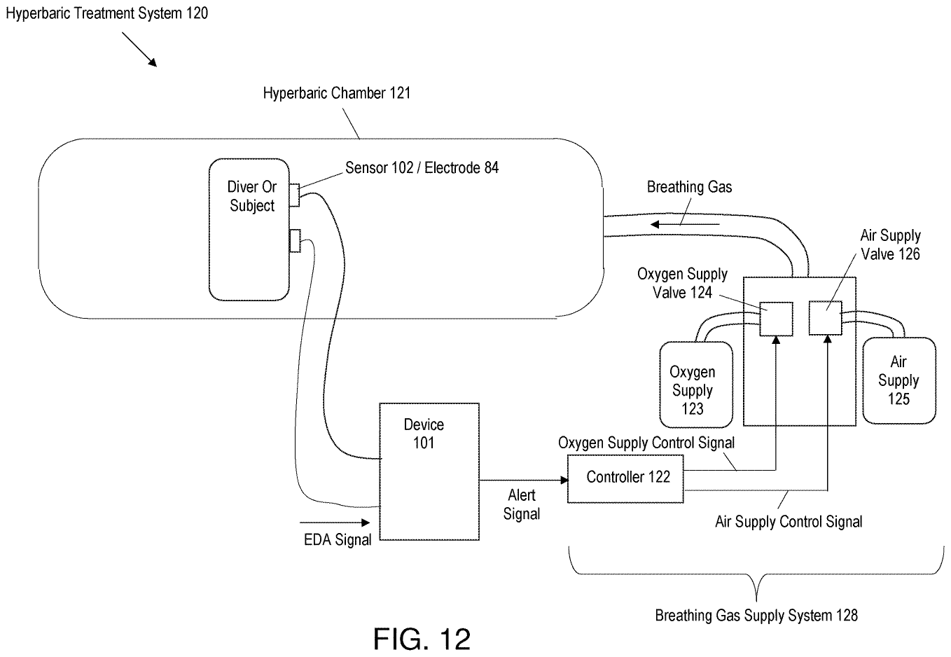

FIG. 12 illustrates a hyperbaric treatment system 120. The hyperbaric treatment system 120 is configured for treating a diver or subject with hyperbaric oxygen in a hyperbaric chamber 121. For a diver being treated for decompression sickness, the hyperbaric chamber 121 may be referred to as a recompression chamber. The hyperbaric treatment system 120 includes the hyperbaric chamber 121, the sensor 102 and/or the electrodes 84, and the device 101. The sensor 102 and/or the electrodes 84 are in contact with the skin of the diver or subject and collect EDA data, which is provided to the device 101 for processing. The sensor 102 and/or the electrodes 84 and the device 101 may be configured as a wearable device similar to the apparatuses illustrated in FIGS. 9 and 10. Alternatively, the device 101 may be disposed outside of the hyperbaric chamber 121 (as shown in FIG. 12) with wires providing collected EDA data to the device 101 through pressure-resistant chamber penetrations (represented where the wires penetrate a wall of the hyperbaric chamber 121).

The device 101 is configured to output an alert signal in response to determining that CNS-OT risk exceeds or meets a threshold of unacceptable risk. The alert signal is output to a breathing gas supply system 128. The breathing gas supply system 128 is configured to supply breathing gas to the diver or subject at a selected partial pressure of oxygen. Upon receiving the alert signal, the breathing gas supply system 128 is configured to reduce the partial pressure of oxygen being delivered to the diver or subject either automatically or by manual interaction by an operator.

The breathing gas supply system 128 includes a controller 122 configured to receive the alert signal and send an oxygen supply control signal to an oxygen supply valve 124 and/or an air supply control signal to an air supply valve 126 in response to receiving the alert signal. The oxygen supply valve 124 is configured to admit oxygen from an oxygen supply 123 to the hyperbaric chamber 121. The air supply valve 126 is configured to admit air from an air supply 125 to the hyperbaric chamber 121. Hence, by decreasing an amount of oxygen and/or increasing an amount of air supplied to the hyperbaric chamber 121, the partial pressure of oxygen being breathed by the diver or subject can be decreased accordingly. Oxygen sensors (not shown) and pressure sensors (not shown) disposed in the breathing gas supply system can sense a partial pressure of oxygen being supplied to the diver or subject and provide feedback to the controller 122 for closed-loop control of the partial pressure of oxygen and maintain a lower oxygen partial pressure setpoint. The breathing gas can be supplied to the diver or subject in an ambient environment in the hyperbaric chamber 121 or by mask that is worn by the diver or subject in the hyperbaric chamber.

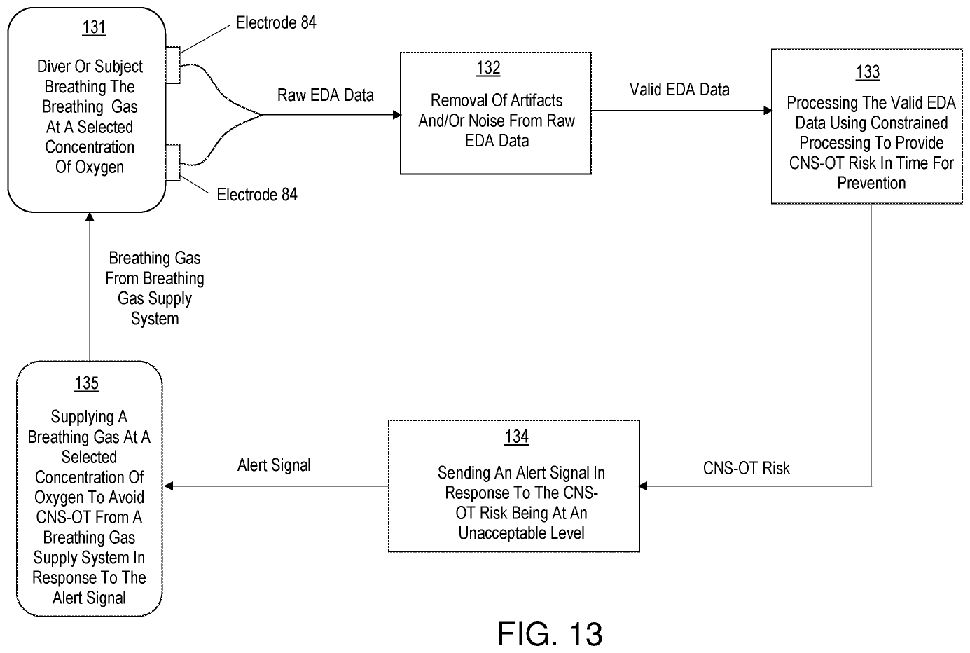

FIG. 13 illustrates closed-loop control of oxygen concentration in a diver or subject. At 131, raw EDA data is obtained from the diver or subject breathing a gas having a first concentration of oxygen using electrodes in contact with the diver or subject. At 132, artifacts and/or noise is removed from the raw EDA data to provide valid EDA data. At 133, the valid EDA data is processed using constrained processing (using the techniques disclosed herein) to provide CNS-OT risk in a timely manner. At 134, an alert signal is sent out in response to the CNS-OT risk being at an unacceptable level. At 135, the concentration of oxygen in the breathing gas of the diver or subject is lowered in response to receiving the alert signal. The closed-loop cycle then repeats itself with obtaining raw EDA data from the diver or subject breathing a gas with the lowered oxygen concentration.

It can be appreciated that the rebreather system 110 may represent any breathing gas supply system for a diver or subject that can have an oxygen concentration setpoint adjusted manually by the diver or automatically by a controller based on a prediction of imminent CNS-OT or a level of CNS-OT risk. In another embodiment, a diver may carry multiple scuba tanks having different concentrations of oxygen. Upon receiving an alert signal related to CNS-OT, the diver can switch to breathing from a scuba tank having a lower concentration of oxygen. In this case, the alert signal influences an action of the diver, which is switching breathing from one scuba tank to another scuba tank having a lower concentration of oxygen to avoid hazards from CNS-OT.

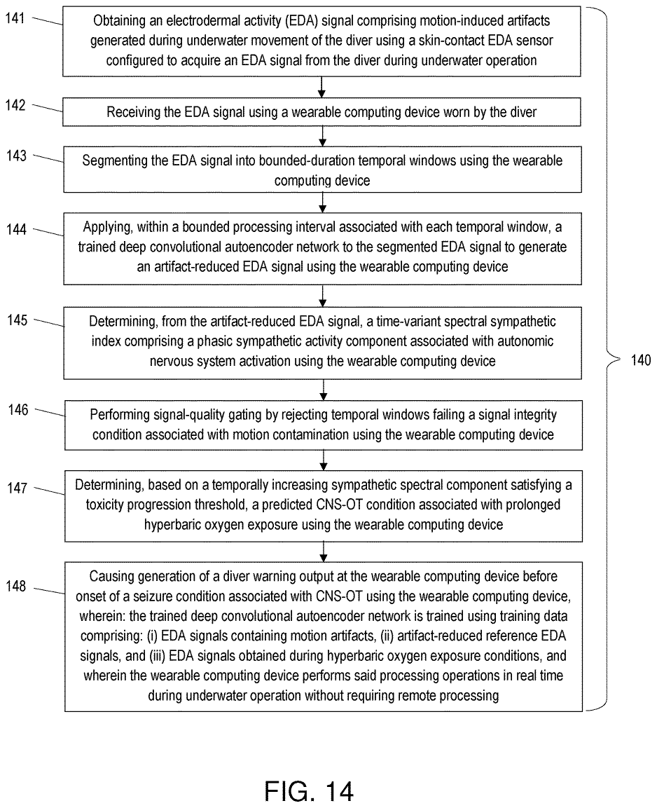

FIG. 14 is a flowchart for a method 130 for physiological monitoring for bounded real-time prediction of central nervous system oxygen toxicity (CNS-OT) of a diver during hyperbaric oxygen exposure. Block 141 calls for obtaining an electrodermal activity (EDA) signal comprising motion-induced artifacts generated during underwater movement of the diver using a skin-contact EDA sensor configured to acquire an EDA signal from the diver during underwater operation. Block 142 calls for receiving the EDA signal using a wearable computing device worn by the diver. The wearable computing device may also include the EDA sensor or multiple EDA sensors.

Block 143 calls for segmenting the EDA signal into bounded-duration temporal windows using the wearable computing device. Block 144 calls for applying, within a bounded processing interval associated with each temporal window, a trained deep convolutional autoencoder network to the segmented EDA signal to generate an artifact-reduced EDA signal using the wearable computing device. In one or more embodiments, the bounded processing interval is less than 5 seconds to allow the diver sufficient time to response to a CNS-OT warning signal.

Block 145 calls for determining, from the artifact-reduced EDA signal, a time-variant spectral sympathetic index comprising a phasic sympathetic activity component associated with autonomic nervous system activation using the wearable computing device. The autonomic nervous system relates to the part of the body's peripheral nervous system that regulates involuntary, unconscious processes without requiring active thought, whereas non-autonomic responses such as bodily movement are not controlled by the autonomic nervous system. Block 146 calls for performing signal-quality gating by rejecting temporal windows failing a signal integrity condition associated with motion contamination using the wearable computing device. In one or more embodiments, signal-quality gating can also include suppressing radiofrequency-induced noise before application of the trained deep convolutional autoencoder network. Radiofrequency noise can be suppressed by knowing the radio frequencies causing the interference and removing those frequency components from the EDA signal. The radio frequencies causing the interference can be determined by analysis of the equipment being used and the environment and/or by testing. In certain cases, temporal windows having radiofrequency interference above a threshold amplitude level can be rejected.

Block 147 calls for determining, based on a temporally increasing sympathetic spectral component satisfying a toxicity progression threshold, a predicted CNS-OT condition associated with prolonged hyperbaric oxygen exposure using the wearable computing device. In one or more embodiments, the toxicity progression threshold is adaptive based on a diver-specific baseline sympathetic spectral profile.

Block 148 calls for causing generation of a diver warning output at the wearable computing device before onset of a seizure condition associated with CNS-OT using the wearable computing device, wherein: the trained deep convolutional autoencoder network is trained using training data comprising: (i) EDA signals containing motion artifacts, (ii) artifact-reduced reference EDA signals, and (iii) EDA signals obtained during hyperbaric oxygen exposure conditions, and wherein the wearable computing device performs said processing operations in real time during underwater operation without requiring remote processing.

In one or more embodiments, the method 130 includes reducing a concentration of oxygen being breathed by the diver from a breathing gas supply system in response to the breathing gas supply system receiving the diver warning output. In one or more embodiments, the breathing gas supply system is a rebreather.

While specific configurations have been described, it is not intended that the scope be limited to the particular configurations set forth, as the configurations herein are intended in all respects to be possible configurations rather than restrictive.

Unless otherwise expressly stated, it is in no way intended that any method set forth herein be construed as requiring that its steps be performed in a specific order. Accordingly, where a method claim does not actually recite an order to be followed by its steps or it is not otherwise specifically stated in the claims or descriptions that the steps are to be limited to a specific order, it is no way intended that an order be inferred, in any respect. This holds for any possible non-express basis for interpretation, including: matters of logic with respect to arrangement of steps or operational flow; plain meaning derived from grammatical organization or punctuation; the number or type of configurations described in the specification.

It will be apparent to those skilled in the art that various modifications and variations may be made without departing from the scope or spirit. Other configurations will be apparent to those skilled in the art from consideration of the specification and practice described herein. It is intended that the specification and described configurations be considered as exemplary only, with a true scope and spirit being indicated by the following claims.

What is claimed is:

1.-20. (canceled)

21. A wearable physiological monitoring system configured for bounded real-time prediction of central nervous system oxygen toxicity (CNS-OT) of a diver during hyperbaric oxygen exposure, the system comprising:
a skin-contact electrodermal activity (EDA) sensor configured to acquire an EDA signal from the diver during underwater operation;
a wearable computing device comprising:
one or more processors;
a non-transitory memory; and
processor-executable instructions that, when executed by the one or more processors, cause the wearable computing device to:
receive the EDA signal comprising motion-induced artifacts generated during underwater movement of the diver;
segment the EDA signal into bounded-duration temporal windows;
apply, within a bounded processing interval associated with each temporal window, a trained deep convolutional autoencoder network to the segmented EDA signal to generate an artifact-reduced EDA signal;
determine, from the artifact-reduced EDA signal, a time-variant spectral sympathetic index comprising a phasic sympathetic activity component associated with autonomic nervous system activation;
perform signal-quality gating by rejecting temporal windows failing a signal integrity condition associated with motion contamination;
determine, based on a temporally increasing sympathetic spectral component satisfying a toxicity progression threshold, a predicted CNS-OT condition associated with prolonged hyperbaric oxygen exposure;
and
cause generation of a diver warning output at the wearable computing device before onset of a seizure condition associated with CNS-OT;
wherein:
the trained deep convolutional autoencoder network is trained using training data comprising:
(i) EDA signals containing motion artifacts,
(ii) artifact-reduced reference EDA signals, and
(iii) EDA signals obtained during hyperbaric oxygen exposure conditions,
and wherein the wearable computing device performs said processing operations at the wearable computing device in real time during underwater operation without requiring remote processing.

22. The system of claim 21, further comprising a breathing gas supply system configured to supply breathing gas at a reduced concentration of oxygen to the diver in response to the diver warning output.

23. The system of claim 22, wherein the breathing gas supply system comprises a controller configured to receive the diver warning output and to provide closed-loop control of the concentration of oxygen being breathed by the diver to avoid a CNS-OT condition.

24. The system of claim 22, wherein the breathing gas supply system is a rebreather system.

25. The system of claim 21, wherein the warning output comprises at least one of a visual output, an audible output, or a vibratory output, each of which being perceptible underwater.

26. The system of claim 21, wherein the bounded processing interval is less than 5 seconds.

27. The system of claim 21, wherein the signal-quality gating comprises rejecting temporal windows exhibiting non-autonomic phasic elevation patterns.

28. The system of claim 21, wherein the trained deep convolutional autoencoder network comprises an encoder path and a decoder path configured to reconstruct artifact-reduced EDA waveforms.

29. The system of claim 21, wherein the wearable computing device is integrated into a dive computer comprising the EDA sensor, a dive watch comprising the EDA sensor, a wrist-mounted wearable device comprising the EDA sensor, a chest-mounted wearable device comprising the EDA sensor, or a diving garment comprising the EDA sensor.

30. The system of claim 21, wherein the toxicity progression threshold is adaptive based on a diver-specific baseline sympathetic spectral profile.

31. The system of claim 21, wherein the bounded-duration temporal windows each comprise between 5 seconds and 60 seconds of EDA data.

32. The system of claim 21, wherein the time-variant spectral sympathetic index is computed using frequency-domain decomposition of phasic EDA activity.

33. The system of claim 21, wherein the wearable computing device is configured to not transmit raw EDA signals to a remote server to reduce transmission bandwidth.

34. The system of claim 21, wherein the wearable physiological monitoring system is configured to operate autonomously and the system further comprises a communications interface for communicating processed data to a remote processing system, the processed data not including raw EDA signals to reduce transmission bandwidth.

35. The system of claim 21, wherein the processor-executable instructions that, when executed by the one or more processors, further cause the wearable computing device to suppress radiofrequency-induced noise before application of the trained deep convolutional autoencoder network.

36. A non-transitory computer-readable media comprising instructions that when executed by a computer implement a method for bounded real-time prediction of central nervous system oxygen toxicity (CNS-OT) in a diver during hyperbaric oxygen exposure, the method comprising:
acquiring, by a wearable electrodermal activity sensor, an electrodermal activity (EDA) signal from the diver during underwater operation;
segmenting the EDA signal into bounded-duration temporal windows;
performing motion-aware artifact reduction on each temporal window using a trained deep convolutional autoencoder network executed locally at a wearable computing device;
rejecting temporal windows that fail a signal-quality condition indicative of excessive motion corruption;
generating, from remaining temporal windows, a time-variant sympathetic spectral measurement comprising a phasic sympathetic activation component;
detecting a temporally progressive increase in the sympathetic spectral measurement relative to a baseline physiological state associated with absence of CNS-OT exposure;
determining, based on the detected increase satisfying a toxicity threshold, a predicted CNS-OT condition;
and
outputting, by the wearable computing device, a diver alert before occurrence of a seizure event.

37. The non-transitory computer-readable media of claim 36, wherein the method further comprises reducing a concentration of oxygen being breathed by the diver from a breathing gas supply system in response to the breathing gas supply system receiving the diver alert.

38. The non-transitory computer-readable media of claim 37, wherein the method further comprises inputting the diver alert into a controller in the breathing gas supply system to provide closed-loop control of the concentration of oxygen being breathed by the diver to avoid a CNS-OT condition.

39. A method for physiological monitoring for bounded real-time prediction of central nervous system oxygen toxicity (CNS-OT) of a diver during hyperbaric oxygen exposure, the method comprising:
obtaining an electrodermal activity (EDA) signal comprising motion-induced artifacts generated during underwater movement of the diver using a skin-contact EDA sensor configured to acquire an EDA signal from the diver during underwater operation;
receiving the EDA signal using a wearable computing device worn by the diver;
segmenting the EDA signal into bounded-duration temporal windows using the wearable computing device;
applying, within a bounded processing interval associated with each temporal window, a trained deep convolutional autoencoder network to the segmented EDA signal to generate an artifact-reduced EDA signal using the wearable computing device;
determining, from the artifact-reduced EDA signal, a time-variant spectral sympathetic index comprising a phasic sympathetic activity component associated with autonomic nervous system activation using the wearable computing device;
performing signal-quality gating by rejecting temporal windows failing a signal integrity condition associated with motion contamination using the wearable computing device;
determining, based on a temporally increasing sympathetic spectral component satisfying a toxicity progression threshold, a predicted CNS-OT condition associated with prolonged hyperbaric oxygen exposure using the wearable computing device;
and
causing generation of a diver warning output at the wearable computing device before onset of a seizure condition associated with CNS-OT using the wearable computing device;
wherein:
the trained deep convolutional autoencoder network is trained using training data comprising:
(i) EDA signals containing motion artifacts,
(ii) artifact-reduced reference EDA signals, and
(iii) EDA signals obtained during hyperbaric oxygen exposure conditions,
and wherein the wearable computing device performs said processing operations in real time during underwater operation without requiring remote processing.

40. The method of claim 39, further comprising reducing a concentration of oxygen being breathed by the diver from a breathing gas supply system in response to the breathing gas supply system receiving the diver warning output.
